(19) United States
(12) Patent Application Publication
(10) Pub. No.: US 2023/0102670 A1
(43) Pub. Date: Mar. 30, 2023

(54) LOW COMPLEXITY LOAD MONITOR

(71) Applicant: T.J.Smith and Nephew,Limited, Hull (GB)

(72) Inventor: Allan Kenneth Frazer Grugeon Hunt, Beverley (GB)

(21) Appl. No.: 17/907,924

(22) PCT Filed: Feb. 26, 2021
(86) PCT No.: PCT/EP2021/054945

§ 371 Date: Aug. 29, 2022

(30) Foreign Application Priority Data
Mar. 3, 2020 (GB) 2003014.4

(51) Int. Cl. A61B 5/103; A61B 5/00

(52) U.S. Cl. CPC A61B 5/1038; A61B 5/6829; A61B 2562/0247; A61B 2560/0204

(57) ABSTRACT

In some aspects, a device is disclosed for monitoring load bearing of a foot. The device can include a housing, a transmitter, and a pressure sensor. The housing can be positioned under a foot of a patient and attach to the foot. The transmitter can wirelessly transmit an indication that a portion of the foot is supporting at least a threshold weight. The pressure sensor can wake up from a sleep mode responsive to the portion supporting at least the threshold weight, cause the transmitter to transmit the indication, and return to the sleep mode.

### CROSS-REFERENCE TO RELATED APPLICATIONS

This application claims priority to U.K. Provisional Application No. 2003014.4 filed on Mar. 3, 2020; the disclosure of which is hereby incorporated by reference in its entirety.

### TECHNICAL FIELD

Embodiments of the present disclosure relate to apparatuses, systems, and methods for preventing or managing wounds with load monitoring.

### BACKGROUND

Pressure ulcers, which may also be known as pressure sores, bedsores, or decubitus ulcers, are injuries to skin and underlying tissue resulting from prolonged pressure on the skin, soft tissue, muscle, or bone above capillary filling pressure (approximately 32 mmHg).

One type of pressure ulcer that develops on a foot is known as a diabetic foot ulcer (DFU), which tends to occur with a higher frequency and intensity in the diabetic population. Management and treatment of diabetic foot ulcers requires offloading the diabetic foot ulcers by using cushioned footwear, such as a support boot, cast, shoe, or the like. While offloading can be effective, it has been found that non-compliance with or non-use of offloading devices can play a large role in the delayed healing of the diabetic foot ulcers.

Prior art approaches and systems provide little or no information regarding an individual's compliance with the offloading devices. Gaining insight into the individual's compliance can be important for the prevention and healing of pressure ulcers. However, because of these limitations, the prevention and healing of pressure ulcers using prior art approaches and systems may be delayed or, worse yet, worsened leading to prolonged discomfort, hospitalization, or even surgery.

### SUMMARY

In some aspects, a device is disclosed for monitoring load bearing of a foot. The device can include a housing, a transmitter, a pressure sensor, and a power source. The housing can be positioned under a foot of a patient and attach to the foot. The transmitter can wirelessly transmit an indication that a portion of the foot is supporting at least a threshold weight. The pressure sensor can wake up from a sleep mode responsive to the portion supporting at least the threshold weight, cause the transmitter to transmit the indication, and return to the sleep mode. The power source can supply power to the transmitter and the pressure sensor.

The device of the preceding paragraph can include one or more of the following features: The pressure sensor can return to the sleep mode within a set period of time after waking up from the sleep mode. The indication can be a burst communication. The housing can enclose the transmitter, the pressure sensor, and the power source. The device can further include an adhesive configured to attach the housing to the foot. The pressure sensor can be initiated responsive to a user input. The housing can be destroyed by detachment of the housing from the foot. The transmitter can transmit the indication responsive to detachment of the housing from the foot. The transmitter can, responsive to detachment of the housing from the foot, repeatedly transmit the indication until an energy level of the power source depletes to a threshold level. The housing can include a gel and a plurality of encapsulation layers. The indication can denote to move the foot. The device can further include a speaker configured to audibly present the indication. The power source can include a battery. The power source can include a piezoelectric element configured to generate power responsive to an application of pressure to the housing. The device can further include: another housing configured to be positioned under the foot and attach to the foot; another transmitter configured to wirelessly transmit another indication that a different portion of the foot is supporting at least another threshold weight; another pressure sensor configured to: wake up from the sleep mode responsive to the different portion supporting at least the another threshold weight, cause the another transmitter to transmit the another indication, and return to the sleep mode; and another power source configured to supply power to the another transmitter and the another pressure sensor. The device can further include a member configured to connect the housing and the another housing. The member can include a conductive path configured to electrically connect the housing and the another housing. The member can control a spacing between the housing and the another housing. The member can stretch. The member can plastically deform to stretch. The member can include a strain gauge configured to activate the pressure sensor and the another pressure sensor. The threshold weight can be different from the another threshold weight.

### BRIEF DESCRIPTION OF THE DRAWINGS

Embodiments of the present disclosure will now be described hereinafter, by way of example, with reference to the accompanying drawings in which:

FIG. 1 illustrates an example load monitor;

FIG. 2 illustrates an example structure for a load monitor, such as the load monitor of FIG. 1;

FIG. 3 illustrates an example monitoring system that includes a load monitor, such as the load monitor of FIG. 1, and a data processing device;

FIG. 4 illustrates an example load monitoring process performable by a load monitor, such as the load monitor of FIG. 1;

FIGS. 5A and 5B illustrate an example two-pad load monitor;

FIGS. 6A and 6B illustrate an example four-pad load monitor; and

FIG. 7 illustrates an example computer system usable to construct one or more of the devices or systems within the monitoring system of FIG. 3.

### DETAILED DESCRIPTION

### Overview

A device is disclosed for monitoring load bearing. The device can have a compact size and form and have limited processing and memory storage capabilities. The device can include and position a pressure sensor proximate to a load bearing surface, such as a body part of an individual, and output a load bearing indication (for instance, a burst wireless communication via electromagnetic radiation or an audible sound via a speaker) to a different device indicating that the load bearing surface experienced a change in loading. The different device can be a bridge device for storing or presenting the load bearing indication or data associated with the load bearing indication (such as time, place, or data input by a user providing information about the load bearing indication). The different device may be worn by or proximate to the load bearing surface or within a communication range from the device. The different device can be a wound therapy device, such as a negative pressure wound therapy device.

The device or its pressure sensor can be in a sleep mode under typical circumstances. Upon the load bearing surface experiencing a change in loading, the device or its pressure sensor may awake so that the device outputs the load bearing indication, and the device or its pressure sensor may subsequently return to the sleep mode. As a result, the device can utilize minimal power to operate, have a long activity or battery life, or incorporate a small or limited power source. The device may largely consume power when preparing for transmission of the load bearing indication and transmitting the load bearing indication and not at other times (such as when the loading of the load bearing surface remains constant or does not change within a threshold amount). The device may poll or be polled by the different device.

The device can include an adhesive, such as an adhesive coating, on a surface of the device that is usable to attach the device to the load bearing surface. The device may not have an adhesive on an opposite surface of the device that may not attached to the load bearing surface. The device can be manufactured to include the adhesive.

The device can be constructed so that detachment of the device from the load bearing surface may destroy the device. Detachment of the device can, for example, tear open a housing of the device deactivating the device or may cause a power supply of the device to disconnect so that the device may no longer be powered.

The device can output a detachment indication responsive to detachment of the device from the load bearing surface. The detachment indication can thus serve to notify of detachment of the device. The device can repeatedly (for example, for a set period of time or instead periodically until an energy level of a power source of the device sufficiently depletes) output the detachment indication responsive to detachment of the device.

The device can include a battery and a communication chip with antenna held between two or more sheet materials. The device can include gel positioned between encapsulation layers to spread a load foci of the battery, the communication chip, or other components of the device.

The device can be powered by a battery (such as a flexible battery or a paper battery), a piezoelectric element, or another type of power source. Where the device is powered by the piezoelectric element, the piezoelectric element may generate charge from pressure to or compression of the device. The charge from the piezoelectric element can be used to directly power one or more components of the device or may instead be used to charge a capacitor, a supercapacitor, or a battery that may buffer the charge prior to use, such as by the pressure sensor. The piezoelectric element can charge from pressure to or compression of the device despite the device not outputting the load bearing indication, such as because the portion of the load bearing surface did not experience a sufficiently large change in loading to trigger the load bearing indication. Detachment of the device from the load bearing surface can cause the piezoelectric element to change its shape, which may generate a charge usable to detect detachment of the device.

The device can include and position multiple pressure sensors, which can be used to monitor different portions of the loading bearing surface (such as different load risk points). The multiple pressure sensors can be mechanically connected by a member, which can include a thermoplastic polyurethane (TPU) that can stretch by plastic deformation and include one or more strain gauges. The member can be reshaped, such as by stretching, to enable the device to be usable for load bearing surfaces of difference sizes or shapes, such as for difference sizes of feet. The multiple pressure sensors can be electrically connected through a member, such as via one or more conductive paths like wires. Pulling one or more of the multiple pressure sensors away from one or more others of the other multiple pressure sensors can initiate the one or more of the multiple pressure sensors or the device. Additionally or alternatively, one or more of the multiple pressure sensors or the device may be initiated by a switch, load cell, magnetic communication, or electromagnetic communication.

The device can be used to indicate if a seal, such as for a wound dressing or another item, has been opened or tampered with. The device can be positioned proximate to or as part of a seal so that the device generates an unseal indication in response to opening, removal, or destruction of the seal. The unseal indication from the device can further be used to manage or audit a warranty for the wound dressing or the another item. In one example, the device can be positioned proximate to a boundary between skin of a patient and a boarder of a wound dressing, and the device can accordingly indicate if therapy was removed.

### Introduction to Load Monitoring

Loading of a body part of an individual may be desirably monitored by a load monitor for a variety of reasons, including wound prevention and monitoring. In one example, loading can be monitored when the individual may be prone to or already have a wound, such as a pressure ulcer. Information generated by the load monitor about the loading can be helpful for assisting with prevention or treatment of the pressure ulcer. In addition, information generated by the load monitor can be useful for checking compliance with a treatment regimen.

Some aspects disclosed herein relate to wound monitoring or therapy for a human or animal body. Therefore, any reference to a wound herein can refer to a wound on a human or animal body, and any reference to a body herein can refer to a human or animal body. The disclosed technology may relate to preventing or minimizing damage to physiological tissue or living tissue, or to the treatment of damaged tissue (for example, a wound as described herein).

As used herein the expression “wound” may include an injury to living tissue that may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound can become a chronic wound when it does not follow the healing stages resulting in a lengthened recovery. It is believed that the transition from acute to chronic wound can be due to a patient being immuno-compromised.

Chronic wounds may include for example: venous ulcers (such as those that occur in the legs), which account for the majority of chronic wounds and mostly affect the elderly, diabetic ulcers (for example, foot or ankle ulcers), peripheral arterial disease, pressure ulcers, or epidermolysis bullosa (EB).

Examples of other wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Wounds may include a deep tissue injury. Deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

Wound may also include tissue at risk of becoming a wound as discussed herein. For example, tissue at risk may include tissue over a bony protuberance (at risk of deep tissue injury/insult) or pre-surgical tissue (for example, knee tissue) that may have the potential to be cut (for example, for joint replacement/surgical alteration/reconstruction).

Some aspects relate to methods of monitoring or treating a wound with the technology disclosed herein in conjunction with one or more of the following: advanced footwear, turning a patient, offloading (such as, offloading diabetic foot ulcers), treatment of infection, systemix, antimicrobial, antibiotics, surgery, removal of tissue, affecting blood flow, physiotherapy, exercise, bathing, nutrition, hydration, nerve stimulation, ultrasound, electrostimulation, oxygen therapy, microwave therapy, active agents ozone, antibiotics, antimicrobials, or the like.

Alternatively or additionally, a wound may be treated using topical negative pressure or traditional advanced wound care, which is not aided by the using of applied negative pressure (may also be referred to as non-negative pressure therapy).

Although the present disclosure may refer to pressure ulcers, foot ulcers, or the like, the systems and methods disclosed herein can be used for monitoring or treating any type of skin injury or wound, such as a venous leg ulcer.

### Load Monitoring Systems and Processes

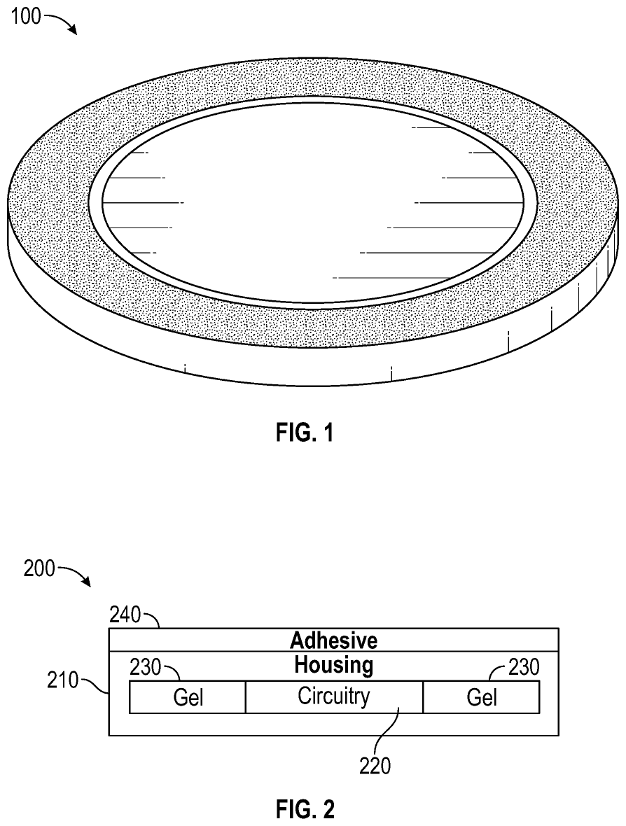

FIG. 1 illustrates a load monitor 100. The load monitor 100 can be disc-shaped and designed for positioning under a load bearing surface, such as a body part of an individual like the bottom of a foot. The load monitor 100 can be attached to the load bearing surface using a strap, adhesive, or other coupling mechanism and may be worn on or supported by the load bearing surface.

The load monitor 100 can emit electromagnetic radiation or a sound to indicate that the load monitor 100 supported at least a threshold weight (for example, 20 N, 40 N, 50 N, 100 N, 150 N, 200 N, 250 N, or 400 N or 5 lbf, 10 lbf, 20 lbf, 30 lbf, 40 lbf, 50 lbf, 75 lbf, or 100 lbf). The electromagnetic radiation or the sound can be a burst communication (for example, a broadcast transmission having a duration of 0.1 sec, 0.2 sec, 0.5 sec, or 1 sec or shorter) and may denote to the individual, a caregiver, or another device that the load monitor 100 supported at least the threshold weight. The threshold weight can be set to be a loading force which risks adversely impacting an existing ulcer or causing a new ulcer. Multiple of the load monitor 100 can be used to monitor the loading of different portions of the load bearing surface or different surfaces.

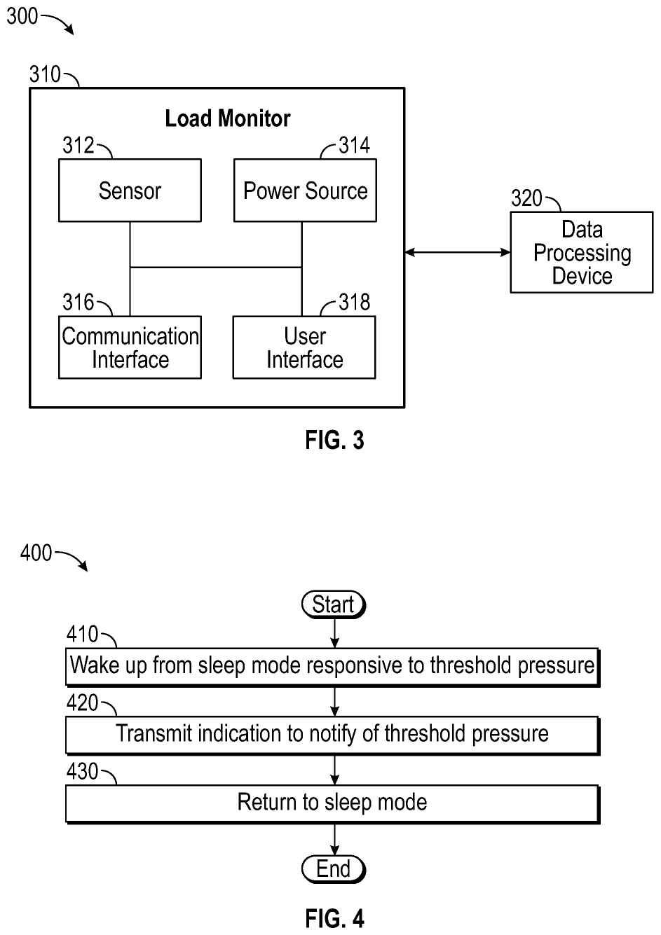

FIG. 2 illustrates an example structure for a load monitor 200, which can be an implementation of the load monitor 100 of FIG. 1. The load monitor 200 can include a housing 210 that supports and encloses circuitry 220 and gel 230. The housing 210 can be composed of a top portion and a bottom portion that are sealed together, and the top portion or the bottom portion can be hard or soft. The gel 230 can spread force or cushion the housing 210 or the circuitry 220 when the housing 210 experiences pressure from supporting weight. The gel 230 can be positioned between encapsulation layers. The load monitor 200 can include an adhesive 240 which may be used to attach the load monitor 200 to a load bearing surface, such as a body part of an individual. The housing can be constructed so that the housing may be destroyed upon detachment of the housing from the load bearing surface.

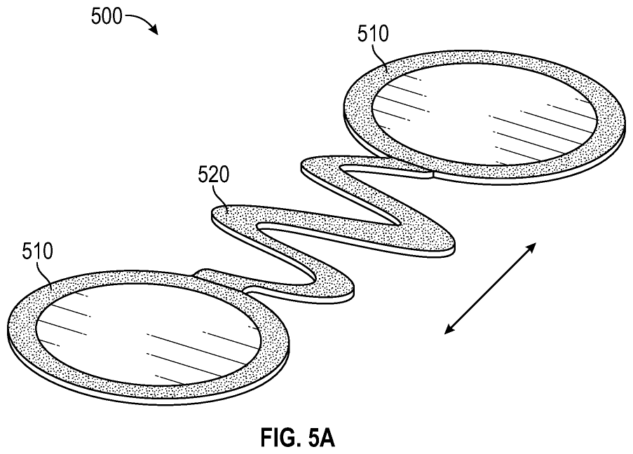

FIG. 3 illustrates a monitoring system 300 that includes a load monitor 310 and a data processing device 320. The load monitor 310 can be an implementation of the load monitor 100 of FIG. 1 or the load monitor 200 of FIG. 2.

The load monitor 310 can include a sensor 312, a power source 314, a communication interface 316, and a user interface 318 that are configured to communicate, such as electrically, with one another. The power source 314 can provide power to one or more components of the load monitor 310.

The sensor 312 can be or include a pressure sensor, which can generate and output a signal when the pressure sensor may support at least a threshold weight. The sensor 312 may additionally in some implementations include one or more other sensors such as accelerometers, gyroscopes, magnetometers, impedance sensors, thermistors, or optical sensors, among other types of sensors. The sensor 312 can include a controller or other circuitry configured to process and control the output of the signal.

The sensor 312, as well as potentially one or more other components of the load monitor 310, can wake up from a sleep mode or enter the sleep mode responsive to one or more conditions. The one or more conditions can, for instance, include the pressure sensor supporting at least the threshold weight, the pressure sensor outputting the signal, the sensor 312 receiving confirmation of receipt or processing of the signal (such as from the communication interface 316). In one example, the sensor 312 can (i) be in a sleep mode when the sensor 312 may not be generating or outputting the signal or awaiting receipt of an expected communication (such as from the communication interface 316) and (ii) awake from the sleep mode to generate or output the signal or await receipt of the expected communication.

The sensor 312 can be in the sleep mode, for example, when a controller of the sensor 312 may be in a sleep mode. The sensor 312 may detect the one or more conditions for controlling the awaking from or entering in sleep mode from an interrupt received by the sensor 312. The sleep mode can be a low power mode of operation for the sensor 312 or other components of the load monitor 310, so the sensor 312 or other components of the load monitor 310 may consume limited or minimal power when in the sleep mode. One or more functions of the sensor 312 or other components of the load monitor 310 can be deactivated or disabled during the sleep mode so that the sensor 312 or the other components of the load monitor 310 may consume the limited or minimal power.

The power source 314 can include one or more of a battery (for instance, a flexibly battery or a paper batter), a piezoelectric element (which can generate power responsive to an application of pressure to the load monitor 310), a capacitor, or a supercapacitor.

The communication interface 316 can include a transmitter. The transmitter can wirelessly transmit (for example, via broadcast radio waves for a duration of 0.1 sec, 0.2 sec, 0.5 sec, or 1 sec or shorter) a load bearing indication that the sensor 312 supported at least the threshold weight. The load bearing indication may denote to move the foot in order to prevent creation or aggravation of a foot ulcer. Moreover, the transmitter can wirelessly transmit (for example, via broadcast radio waves for a duration of 0.1 sec, 0.2 sec, 0.5 sec, or 1 sec or shorter) a detachment indication that the load monitor 310 has been detached from a load bearing surface. The detachment indication can be transmitted repeatedly (for example, periodically), such as until an energy level of the power source 314 depletes to a threshold level. The detachment indication may notify of detachment of the load monitor 310 from the load bearing surface.

The load bearing indication or the detachment indication can be received and processed by the data processing device 320, such as for storage, forwarding to another device (such as to a caregiver device where the data processing device 320 may be a patient device), or presentation via a user notification. The data processing device 320 may enable a patient or a caregiver to review the load bearing indication or the detachment indication from the load monitor 310 and may provide additional information or context (such as a time, a location, or other information associated with an indication occurrence) for understanding the load bearing indication or the detachment indication. The load bearing indication or the detachment indication can, in some implementations, be the same type of transmission by the load monitor 310 so that the data processing device 320 may be unable to distinguish the load bearing indication or the detachment indication from the received indications without utilizing other information (for example, a repetition or timing of a received indication) to distinguish the received indications. The data processing device 320 can be a personal computing device (such as a smart phone, a tablet computer, a wrist-worn activity monitor, or a desktop computer) or a therapy device (such as a negative pressure wound therapy device).

The communication interface 316 may, in some aspects, be unable to successfully communicate farther than 10 meters, 30 meters, or 100 meters away from the communication interface 316. The communication interface 316 may or may not include a receiver that wirelessly receives information.

The user interface 318 can include one or more input elements, such as buttons, switches, dials, touch pads, magnetic field detectors, photodetectors, or microphones, for receiving user inputs for initiating (for example, powering on) or configuring the load monitor 310. In some aspects, the user interface 318 may have no more than one or two user input elements for receiving user inputs. The user interface 318 can include one or more output elements, such as visual feedback devices (for example, light emitting diodes), haptic feedback devices, or audio devices (for example, speakers), that provide user outputs. The one or more output elements can convey status information like whether the load monitor 310 was activated or whether the sensor 312 generated and outputted the signal.

In some aspects, one or more of the sensor 312, the power source 314, the communication interface 316, or the user interface 318 can be at least part of the circuitry 220 of the load monitor 200 of FIG. 2.

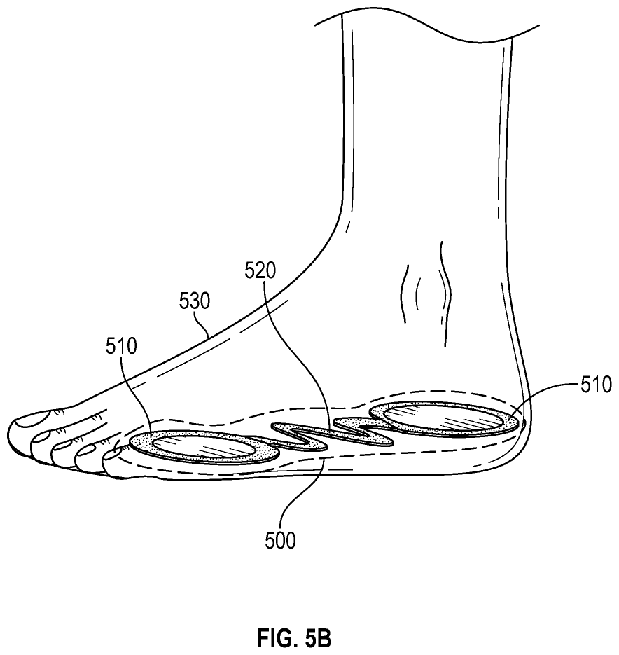

FIG. 4 illustrates a load monitoring process 400 performable by a load monitor, such as the load monitor 100 of FIG. 1, the load monitor 200 of FIG. 2, or the load monitor 310 of FIG. 3. For convenience, the load monitoring process 400 is described in the context of the monitoring system 300, but may instead be implemented in other devices or systems described herein, or by other computing systems not shown. The load monitoring process 400 can advantageously, in certain embodiments, enable the load monitor 310 to function with limited components and complexity and yet notify the data processing device 320 or an individual of excessive loading of a body part which the load monitor 310 is monitoring.

At block 410, the load monitoring process 400 can cause waking up from a sleep mode responsive to a threshold pressure. For example, the sensor 312 of the load monitor 310 can wake up responsive to the threshold pressure (such as a threshold weight) being applied to the sensor 312, and the sensor 312 may generate and output the signal indicative of the threshold pressure being applied to the sensor 312.

At block 420, the load monitoring process 400 can transmit an indication to notify of the threshold pressure. For example, the communication interface 316 of the load monitor 310 can transmit an indication to the data processing device 320 that the threshold pressure was applied to the sensor 312

At block 430, the load monitoring process 400 can return to the sleep mode. For example, the sensor 312 can return to a sleep mode once the signal may be output by the sensor 312, the indication may be transmitted by the communication interface 316, or within a set time thereafter (such as 0.1 sec, 0.2 sec, 0.3 sec, 0.5 sec, 0.7 sec, or 1 sec).

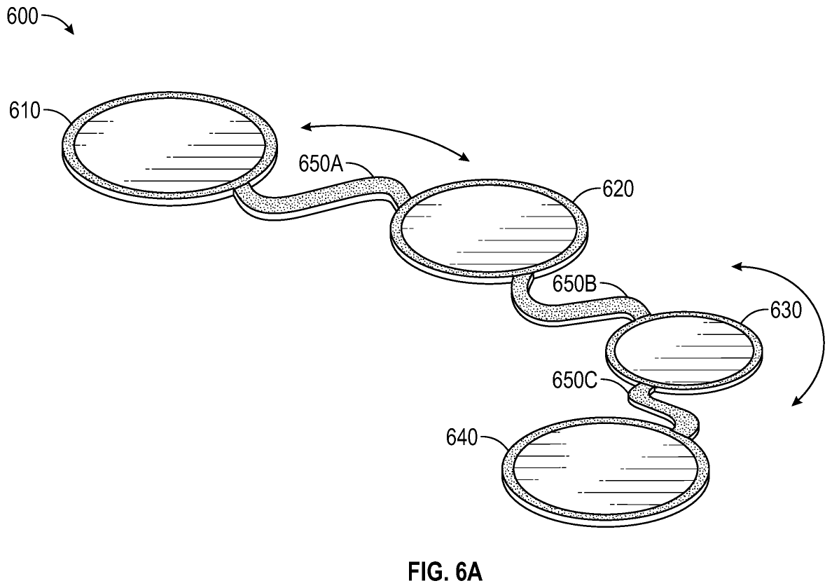

FIGS. 5A and 5B illustrate a two-pad load monitor 500. The two-pad load monitor 500 can include pads 510 and a connector member 520. Each of the pads 510 can be an implementation of the load monitor 100 of FIG. 1, the load monitor 200 of FIG. 2, or the load monitor 310 of FIG. 3. The pads 510 can be positioned proximate to or attached on different portions of a load bearing surface, such as a bottom of a foot 530 as illustrated in FIG. 5B, so that each of the pads 510 can monitor loading of different portions of the load bearing surface. One of the pads 510 may transmit a load bearing indication when a first weight is supported, and the other of the pads 510 may transmit a load bearing indication when a second weight is supported. The first weight can be the same or different from the second weight.

The connector member 520 can mechanically and electrically connect the pads 510. The connector member 520 can control a spacing between the pads 510 and may physically deform to stretch and adjust the spacing. The connector member 520 can include a thermoplastic polyurethane (TPU). The connector member can include a conductive path (for example, wiring) that electrically connects the pads 510. The connector member 520 can include one or more strain gauges that can detect pulling of one of the pads 510 away from the other and be used to initiate (for example, power on) one or both of the pads 510. Once initiated, in some aspects, each of the pads 510 can function independently of one another.

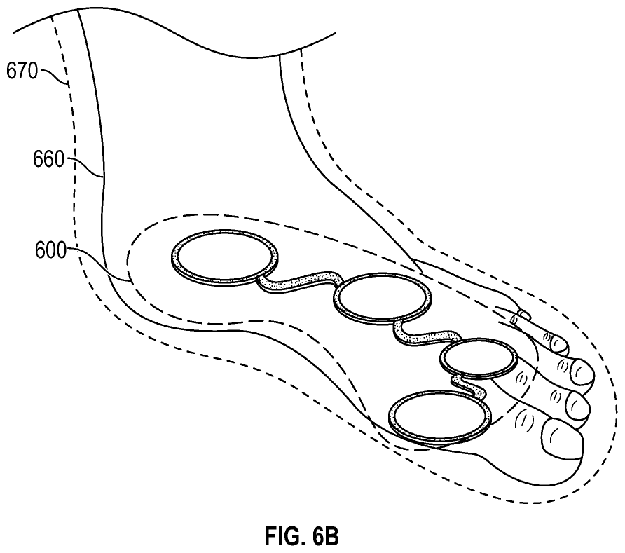

FIGS. 6A and 6B illustrate a four-pad load monitor 600. The four-pad load monitor 600 can include a first pad 610, a second pad 620, a third pad 630, a fourth pad 640, a first connector member 650A, a second connector member 650B, and a third connector member 650C. Each of the first pad 610, the second pad 620, the third pad 630, and the fourth pad 640 can be an implementation of the load monitor 100 of FIG. 1, the load monitor 200 of FIG. 2, or the load monitor 310 of FIG. 3.

The first pad 610, the second pad 620, the third pad 630, and the fourth pad 640 can be positioned proximate to or attached on different portions of a load bearing surface, such as a bottom of a foot 660 and between the foot 660 and a cast 670 (for example, a total contact casting (TCC)) as illustrated in FIG. 6B, so that each of the first pad 610, the second pad 620, the third pad 630, and the fourth pad 640 can monitor loading of different portions of the load bearing surface. One or more of the first pad 610, the second pad 620, the third pad 630, and the fourth pad 640 may transmit a load bearing indication when a common weight or a different weight may be supported, so one or more of the first pad 610, the second pad 620, the third pad 630, and the fourth pad 640 may be triggered by supporting the same or different weights.

The first connector member 650A, the second connector member 650B, and the third connector member 650C can mechanically and electrically connect the first pad 610, the second pad 620, the third pad 630, and the fourth pad 640 as illustrated in FIGS. 6A and 6B. The first connector member 650A, the second connector member 650B, and the third connector member 650C can control a spacing between the first pad 610, the second pad 620, the third pad 630, and the fourth pad 640 and may have the same or different lengths from one another and can separately physically deform to stretch and adjust the spacing. The first connector member 650A, the second connector member 650B, or the third connector member 650C can include a thermoplastic polyurethane (TPU).

One or more of the first connector member 650A, the second connector member 650B, and the third connector member 650C can include a conductive path (for example, wiring) that electrically connects one or more of the first pad 610, the second pad 620, the third pad 630, and the fourth pad 640. One or more of the first connector member 650A, the second connector member 650B, and the third connector member 650C can include one or more strain gauges that may detect pulling of one of the first pad 610, the second pad 620, the third pad 630, and the fourth pad 640 away from another and be used to initiate (for example, power on) one or more or all of the first pad 610, the second pad 620, the third pad 630, and the fourth pad 640. Once initiated, in some aspects, each of the first pad 610, the second pad 620, the third pad 630, and the fourth pad 640 can function independently of one another.

### Computer System Components

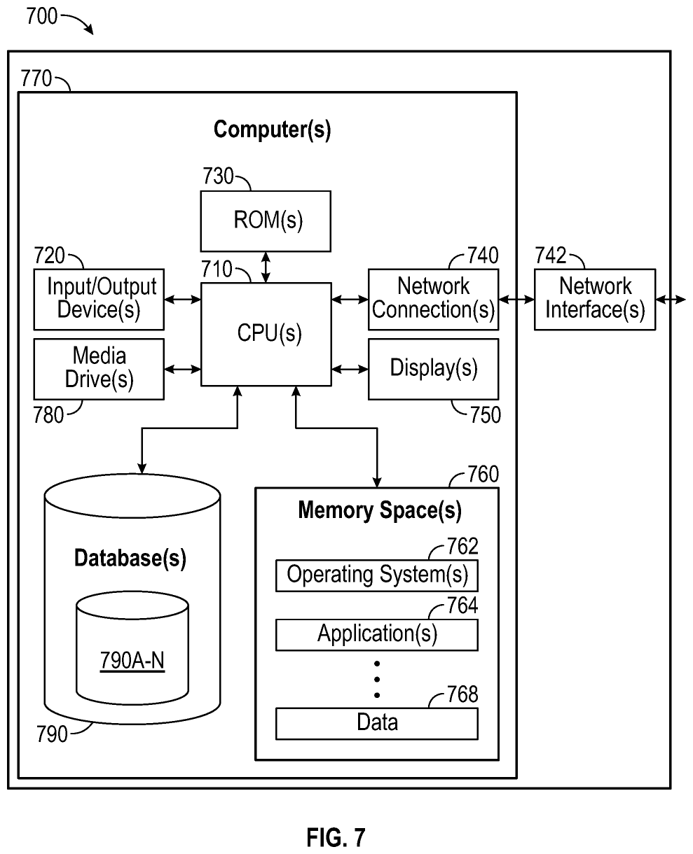

FIG. 7 illustrates a computer system 700 usable to construct one or more of the devices (for instance, the data processing device 320), systems, servers, or the like within the monitoring system 300 of FIG. 3.

As shown in FIG. 7, the computer system 700 can include (i) a processor(s) (CPUs) 710, (ii) an input/output device(s) 720 configured to allow users to input and output information and interact with the computer system 700 as well as transfer and receive data or capture data with one or more sensors like an image sensor, (iii) a read only memory device(s) (ROMs) 730 or equivalents to provide nonvolatile storage of data or programs, (iv) a display(s) 750 such as a computer monitor or other display device, (v) a network connection(s) 740 and a network interface(s) 742 configured to allow the computer system 700 to connect to other systems, servers, or portable devices, as well as a memory space(s) 760 and a database(s) 790. The database(s) 790 may be further divided or distributed as sub-database(s) 790A-790N, with the sub-database(s) storing feature or function specific information associated with a particular feature or function. The various components shown in FIG. 7 may be incorporated in a computer(s) 770. It is noted that the various components shown in FIG. 7, including the database(s) 790, are typically included as part of the computer(s) 770, however, they may be external to the computer(s) 770 in some aspects. For example, the database(s) 790 may be external to the computer(s) 770 and may be part of a separate database computer system or networked database system. In some instances, the computer system 700 may be a computing device like a desktop computer, mobile phone, or a server.

The memory space(s) 760 may include DRAM, SRAM, FLASH, hard disk drives, or other memory storage devices, such as a media drive(s) 780, configured to store an operating system(s) 762, an application program(s) 764, and data 768, and the memory space(s) 760 may be shared with, distributed with or overlap with the memory storage capacity of the database(s) 790. In some aspects, the memory space(s) 760 may include the database(s) 790 or in some aspects the database(s) 790 may include the data 768 as shown in the memory space(s) 760. The data stored in the memory space(s) 760 or the database(s) 790 may include information, such as indication data, data processing routines, or other types of data described herein.

### Other Variations and Terminology

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, a microprocessor, a state machine, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A hardware processor can include electrical circuitry or digital logic circuitry configured to process computer-executable instructions. In another aspect, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language used herein, such as, among others, “can,” “might,” “may,” “e.g.,” and the like, unless specifically stated otherwise, or otherwise understood within the context as used, is generally intended to convey that certain aspects include, while other aspects do not include, certain features, elements or states. Thus, such conditional language is not generally intended to imply that features, elements or states are in any way required for one or more aspects or that one or more aspects necessarily include logic for deciding, with or without author input or prompting, whether these features, elements or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others.

1. A device for monitoring load bearing of a foot, the device comprising:
a housing configured to be positioned under a foot of a patient and attach to the foot;
a transmitter configured to wirelessly transmit an indication that a portion of the foot is supporting at least a threshold weight;
a pressure sensor configured to:
wake up from a sleep mode responsive to the portion supporting at least the threshold weight,
cause the transmitter to transmit the indication, and
return to the sleep mode within a set period of time after waking up from the sleep mode; and
a power source configured to supply power to the transmitter and the pressure sensor.

2. The device of claim 1, wherein the indication is a burst communication.

3. The device of claim 1, wherein the housing is configured to enclose the transmitter, the pressure sensor, and the power source.

4. The device of claim 1, further comprising an adhesive configured to attach the housing to the foot.

5. The device of claim 1, wherein the pressure sensor is initiated responsive to a user input.

6. The device of claim 1, wherein the housing is configured to be destroyed by detachment of the housing from the foot.

7. The device of claim 1, wherein the transmitter is configured to transmit the indication responsive to detachment of the housing from the foot.

8. The device of claim 7, wherein the transmitter is configured to, responsive to detachment of the housing from the foot, repeatedly transmit the indication until an energy level of the power source depletes to a threshold level.

9. The device of claim 1, wherein the housing comprises a gel and a plurality of encapsulation layers.

10. The device of claim 1, wherein the indication denotes to move the foot.

11. The device of claim 1, further comprising a speaker configured to audibly present the indication.

12. The device of claim 1, wherein the power source comprises a battery.

13. The device of claim 1, wherein the power source comprises a piezoelectric element configured to generate power responsive to an application of pressure to the housing.

14. The device of claim 1, further comprising:
another housing configured to be positioned under the foot and attach to the foot;
another transmitter configured to wirelessly transmit another indication that a different portion of the foot is supporting at least another threshold weight;
another pressure sensor configured to:
wake up from the sleep mode responsive to the different portion supporting at least the another threshold weight,
cause the another transmitter to transmit the another indication, and
return to the sleep mode; and
another power source configured to supply power to the another transmitter and the another pressure sensor.

15. The device of claim 14, further comprising a member configured to connect the housing and the another housing.

16. The device of claim 15, wherein the member comprises a conductive path configured to electrically connect the housing and the another housing.

17. (canceled)

18. The device of claim 15, wherein the member is configured to stretch.

19. The device of claim 18, wherein the member is configured to plastically deform to stretch.

20. The device of claim 15, wherein the member comprises a strain gauge configured to activate the pressure sensor and the another pressure sensor.

21. The device of claim 14, wherein the threshold weight is different from the another threshold weight.

22. (canceled)
